(19) United States
(12) Patent Application Publication
(10) Pub. No.: US 2025/0186103 A1
(43) Pub. Date: Jun. 12, 2025

(54) HANDPIECES FOR USE WITH ELECTROCAUTERY DISSECTING DEVICES AND SYSTEMS

(71) Applicant: MODULE MED LLC, Sugar Land, TX (US)

(72) Inventor: Kevin Zhenmei LIAO, Sugar Land, TX (US)

(73) Assignee: MODULE MED LLC, Sugar Land, TX (US)

(21) Appl. No.: 19/054,730

(22) Filed: Feb. 14, 2025

Related U.S. Application Data
(63) Continuation-in-part of application No. 18/644,578, filed on Apr. 24, 2024, now Pat. No. 12,226,144.
(60) Provisional application No. 63/607,459, filed on Dec. 7, 2023.

(51) Int. Cl. A61B 18/14; A61B 18/00

(52) U.S. Cl. CPC A61B 18/14; A61B 2018/00077; A61B 2018/00083; A61B 2018/00595; A61B 2018/0066; A61B 2018/0072; A61B 2018/00744; A61B 2018/00916; A61B 2018/0094

(57) ABSTRACT

An electrocautery handpiece is provided. The electrocautery handpiece includes an electrocautery device, a housing, one or more electricity flow controllers operably coupled to an electricity control unit for controlling the flow to the electrocautery device, and one or more gas flow controllers for controlling the gas flow through the gas delivery channel of the electrocautery device. In another aspect, a surgical device for the controlled dissection of biological tissue is provided.

### CROSS-REFERENCE TO RELATED APPLICATIONS

This application is a continuation-in-part application of U.S. patent application Ser. No. 18/644,578, filed Apr. 24, 2024, which application claims priority to U.S. Provisional Patent Application. No. 63/607,459, filed Dec. 7, 2023, the entirety of which is incorporated herein by reference.

### FIELD

Embodiments of the present disclosure are directed to an electrocautery handpiece for use with electrocautery devices for and systems for dissecting biological tissue. The devices and methods are generally applicable in healthcare settings and, more particularly, in surgical settings.

### BACKGROUND

Electrocautery devices are used to dissect, cut, coagulate, desiccate, and/or fulgurate biological tissue by applying an electric current to the tissue via an electrocautery tip on the device. Electrocautery devices may be operated using a cautery handpiece which, when connected to an energy source (such as an electrical generator), allow a user to selectively vary the amount of power provided to the electrocautery device from the energy source, thereby facilitating controlled dissection of tissue. Electrocautery devices currently known in the art are not reliably safe when used near fragile and important structures such as vessels and nerves.

During surgical operations, known electrocautery tips can cause vessels to become injured during cutting and dissecting in the tissue if the tip is touching the vessel directly or in too close of proximity to the vessels. Additionally, the effectiveness of existing electrocautery devices can be adversely affected by factors such as pooled body fluid (e.g., blood) in the surgical field and char build-up on the electrocautery tip. Further, known electrocautery devices generate smoke which can reduce visibility in a surgical setting and have the potential to cause fires in high-oxygen operative fields such as the head, neck, and chest areas where oxygen is delivered to a patient.

Accordingly, there is a need for electrocautery devices and methods of use that can be safely and effectively used around fluids, do not cause tissue injury, improve surgical field visibility, and mitigate fire risks.

### BRIEF SUMMARY

In a first aspect, an electrocautery device for dissecting biological tissue is provided. The electrocautery device comprises an insulated electrode and an electrocautery device body. The insulated electrode of the electrocautery device comprises an electrode having a distal tip configured to deliver an electrical current to a biological tissue and a proximal end configured to be coupled to an energy supply source and an insulation material radially disposed on only a middle portion of the electrode such that the distal tip and the proximal end of the electrode is not covered by the insulation material. The electrocautery device body of the electrocautery device is formed from a device body material and comprises a lower portion radially disposed upon the insulated electrode and an upper portion formed as a single unitary piece with the lower portion. The upper portion defines a gas delivery assembly comprising a first portion formed at the proximal end of the upper portion and a second portion formed at the distal end of the upper portion and adjacent to the first portion. The first portion of the upper portion comprises a gas supply connection that is configured to be coupled to a gas supply source. The second portion defines a gas delivery channel having a gas tip, wherein the gas delivery channel is in fluid communication with the gas supply connection such that, when a gas supply source is coupled to the gas supply connection, the gas delivery channel is configured to deliver a gas provided by the gas supply source to a biological tissue via the gas tip.

In some embodiments, the insulated electrode is formed by overmolding the insulation material onto a middle portion of the electrode. In some such embodiments, the insulation material may be overmolded onto a middle portion of the electrode by injection molding. In certain embodiments, the electrocautery device body is formed by overmolding the device body material onto the insulated electrode. In some such embodiments, the insulation material is overmolded onto a middle portion of the electrode by injection molding. In some embodiments, the insulation material is overmolded onto a middle portion of electrode by compression molding. In some embodiments, the insulation material can be combined by using adhesive to bond onto the middle portion of the electrode.

In some embodiments, the distal tip of the electrode is shaped as a blade. In some embodiments, the gas tip is formed from a third material that is distinct from the insulation material and the device body material. In certain embodiments, the gas tip is disposed adjacent to the distal tip of the insulated electrode. The gas tip may contact the distal tip at least one location to facilitate preferred gas dispersion patterns.

In some embodiments, the gas supply connection is a threaded connection. Additionally or alternatively, the gas supply connection may be a tapered connection configured to facilitate a friction fit between the gas supply source and the gas supply connection.

In some embodiments, the insulated electrode further comprises a buffer disposed on an external surface of the insulation material and configured to mitigate or prevent deformation of the insulation material. In such embodiments, the buffer may optionally comprise one or more lugs on at least one end of the buffer and the insulation material comprises anti-rotation tabs. In such embodiments the lugs and the anti-rotation tabs are configured to interact such that the buffer is held in place by the anti-rotation tabs.

In some embodiments, the proximal end of the electrode is configured to be coupled to a handpiece that is operably coupled to the energy supply source and configured to, when manipulated by a user, increase or decrease an amount of power provided to the electrocautery device by the electrical current.

In some embodiments, the second portion of the upper portion of the electrocautery device body and the lower portion of the electrocautery device body form an acute angle. In some embodiments, the second portion of the upper portion of the electrocautery device body and the lower portion of the electrocautery device body is canted such that the angle between its proximal end and the lower portion of the device body is less than the angle between its distal end and the lower portion of the device body. In other embodiments, the second portion of the upper portion of the electrocautery device body and the lower portion of the electrocautery device body is canted such that the angle between its proximal end and the lower portion of the device body is greater than the angle between its distal end and the lower portion of the device body.

In a second aspect, a system for dissecting biological tissue is provided. The system comprises an electrocautery device, a gas supply source coupled to a gas supply of the electrocautery device via a gas supply tube, an energy supply source configured to deliver an electrical current to the electrode of the electrocautery device, and a handpiece that is operably coupled to the energy supply source. In some embodiments, the gas supply tube is permanently coupled to the gas supply connection of the electrocautery device via overmolding. In some embodiments, the gas supply tube is permanently coupled to the gas supply connection of the electrocautery device with adhesive.

The electrocautery device of the system for dissecting biological tissue comprises an insulated electrode and an electrocautery device body. The insulated electrode of the electrocautery device comprises an electrode having distal tip configured to deliver an electrical current to a biological tissue and a proximal end configured to be coupled to an energy supply source and an insulation material radially disposed on only a middle portion of the electrode such that the distal tip and the proximal end of the electrode is not covered by the insulation material. The electrocautery device body of the electrocautery device is formed from a device body material and comprises a lower portion radially disposed upon the insulated electrode and an upper portion formed as a single unitary piece with the lower portion. The upper portion defines a gas delivery assembly comprising a first portion formed at the proximal end of the upper portion and a second portion formed at the distal end of the upper portion and adjacent to the first portion. The first portion of the upper portion comprises a gas supply connection that is configured to be coupled to a gas supply source. The second portion defines a gas delivery channel having a gas tip, wherein the gas delivery channel is in fluid communication with the gas supply connection such that, when a gas supply source is coupled to the gas supply connection, the gas delivery channel is configured to deliver a gas provided by the gas supply source to a biological tissue via the gas tip.

In some embodiments, the system further comprises a gas pump operably coupled to a proximal end of the gas tube and configured to facilitate movement of a gas from the gas source to the gas delivery assembly of the electrocautery device. In some such embodiments, the gas tube is secured to the wire of the handpiece via a coupler.

In a third aspect, an electrocautery handpiece is provided. The electrocautery handpiece comprises an electrocautery device, a housing defining an internal cavity configured to at least partially receive the electrocautery device, one or more gas flow controllers, and one or more electricity flow controllers operably coupled to an electricity control unit. The gas flow controller(s) have an external portion that extends through a gas flow controller opening in the housing and an internal portion that is disposed within the cavity of the housing may be used to selectively restrict a gas flow through a gas delivery channel of the electrocautery device. The electricity flow controller(s) have an external portion that extends through an electricity flow controller opening in the housing and an internal portion that is disposed within the internal cavity of the housing. When the internal portion of the electricity flow controller(s) are actuated by a user, the electricity control unit is configured to supply electricity to the electrocautery device.

In some embodiments, the gas flow controller(s) decrease the gas flow through the gas delivery channel by exerting pressure on the gas delivery channel of the electrocautery device when the external portion of the gas flow controller(s) are actuated in a first direction. In some such embodiments, the gas flow controller(s) prohibits the flow of gas through the gas delivery channel when the external portion is fixed in a first position and allows the flow of gas through the gas delivery channel when the external portion of the gas flow controller is fixed in a second position. In other embodiments, the gas flow through the gas delivery channel is varied by modifying the distance of the external portion of the gas flow controller(s). In some embodiments, the external portion of a gas flow controller is a roller.

In some embodiments, the gas flow controller opening(s) are sized and/or dimensioned to limit actuation or manipulation of the gas flow controller(s) in the first direction such that the gas flow controller is prevented from precluding gas flow through the gas delivery channel (either in whole or in part). In some embodiments, the one or more of the gas flow controllers have an occlusive element for occluding the gas delivery channel in response to actuation and/or manipulation of the gas flow controller(s) such that the gas flow through the gas delivery channel is decreased. In some embodiments, the gas controller is located outside of the electrocautery handpiece.

In some embodiments, the electrocautery handpiece further comprises a pressure relief valve operably coupled to the gas delivery channel of the electrocautery device. In some embodiments, the gas delivery channel is a gas tube. In some such embodiments, the internal portion of the electricity flow controller(s) define an opening dimensioned to allow the gas tube to extend therethrough.

In some embodiments, the housing of the electrocautery handpiece may have fittings disposed in its internal cavity. In such embodiments, one or more components of the electrocautery device may be couplable to such fittings. The electrocautery handpiece (or any portion thereof) may be coupled or permanently fixed to the electrocautery device (or any portion thereof).

In some embodiments, the electricity flow controller(s) include buttons that are actuatable by a user to cause the electrical control unit to supply electricity to the electrocautery device. In some embodiments, the external portion of the electricity flow controller(s) includes an electricity control mechanism that allows the user to adjust the amount of electricity supplied to the electrocautery device.

In a fourth aspect, a surgical device for controlled dissection of biological tissue comprising an electrocautery device operably coupled to an electrocautery handpiece is provided. The electrocautery device includes an insulated electrode having a distal tip configured to deliver an electrical current to a biological tissue and a gas delivery assembly having a gas delivery channel and a gas tip. The gas tip is coupled to and extends distally from the gas delivery channel, which is operably couplable to a gas supply source. The electrocautery handpiece of the surgical device includes a housing, an electricity control unit, one or more electricity flow controllers and one or more gas flow controllers. The electricity flow controller(s) are operably coupled to the electricity control unit in a way that allows the flow of electricity to the electrocautery device to be varied via the electricity flow controller(s) and the gas flow controller(s) are operably coupled to the gas delivery assembly of the electrocautery device in a way that allows the flow of gas in the gas delivery channel to be varied via the gas flow controller(s).

### BRIEF DESCRIPTION OF THE SEVERAL VIEWS OF THE DRAWING(S)

In order to understand and to see how the present disclosure may be carried out in practice, examples will now be described, by way of non-limiting examples only, with reference to the accompanying drawings, in which:

FIG. 1A illustrates an electrode for use in an electrocautery device.

FIG. 1B illustrates an electrode having a layer of insulating material disposed thereon.

FIG. 1C illustrates an isometric view of the insulated electrode of FIG. 1B.

FIG. 2 illustrates an example of the electrocautery device.

FIG. 3 illustrates an additional view of the electrocautery device of FIG. 2.

FIG. 4 illustrates another additional view of the electrocautery device of FIG. 2.

FIG. 5 illustrates an example of the electrocautery device in which a section of the insulation material is recessed to define notches.

FIG. 6 illustrates a buffer configured for placement around the insulation material.

FIG. 7A illustrates an insulated electrode having insulation material shaped to define anti-rotation tabs for connection with a buffer.

FIG. 7B illustrates an electrode shaped to form the anti-rotation tabs of FIG. 7A.

FIG. 8 illustrates an electrocautery device having a buffer disposed around the insulation material.

FIG. 9 illustrates an example of the electrocautery device in which the distal end of the gas delivery assembly is formed from a different material.

FIG. 10 illustrates an example of the electrocautery device in which the distal end of the gas delivery assembly has been canted.

FIG. 11 illustrates an example of the electrocautery device further comprising a gas supply tube.

FIG. 12 illustrates an example of the electrocautery device having an extended length.

FIG. 13 illustrates an example of the electrocautery device having an extended length and canted distal tip.

FIG. 14 illustrates an example electrosurgical handpiece for use with the electrocautery device.

FIG. 15 illustrates an example of the electrocautery device coupled to an electrosurgical handpiece.

FIG. 16 illustrates a front perspective view of an example electrosurgical handpiece for use with the electrocautery device.

FIG. 17 illustrates a side view of the internal components of the electrosurgical handpiece of FIG. 16.

FIG. 18 illustrates a side view of the buttons of FIGS. 16 and 17.

FIG. 19 illustrates an example of the system for dissecting biological tissue.

FIG. 20 illustrates a handpiece with a gas aperture for controlling gas flow through the gas delivery channel.

### DETAILED DESCRIPTION

The invention now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

### Definitions

As used herein, the terms “coupled” and “operably coupled” may refer to one or more components being electrically, mechanically, thermally, chemically, or otherwise linked to another component(s). For example, components may be part of the same structure and/or integral with one another (i.e., “directly coupled”). In other examples, components may be connected via remote means (e.g., via a signal transmitted to an electronic circuit) or voice controlled.

As used herein, the term “dissect” (and all of its derivative forms) may refer to dissecting, cutting, desiccating, and/or fulgurating tissue as well as the coagulation of biological fluids.

As used herein, the term “overmold” (and all of its derivative forms) may refer to any process by which a material is molded over a base substrate, such as, by way of non-limiting example, compression molding or injection molding.

The present invention is an electrocautery device comprising a low-profile gas delivery assembly configured to simultaneously deliver a gas while dissecting biological tissue, thereby reducing the generation of smoke and potential fire hazard by reducing oxygen concentrations in the surgical area, keeping the operative field dry, clearing debris, and increasing visibility of the surgical site.

The electrocautery device is constructed by coating the middle portion of an electrode with an insulation material to form an insulated electrode. The distal tip of the electrode remains exposed for contacting tissue and the proximal end of the electrode is exposed for attachment to an electrosurgical handpiece. The insulated electrode is then overmolded with a device body material to form an electrocautery device body. Optionally, a buffer may be added between the insulation material and the device body material to prevent deformation of the insulation material during the overmolding process.

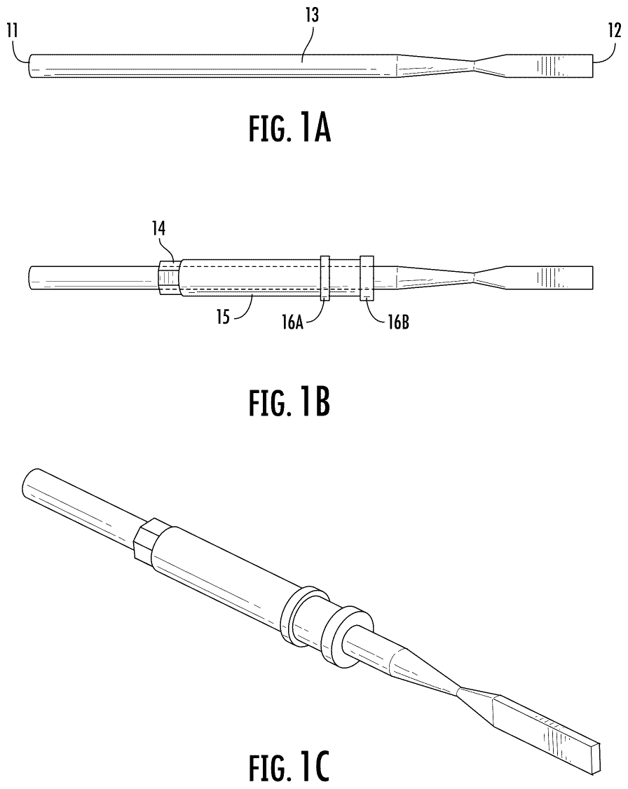

With reference to FIG. 1A, an electrode 13 is illustrated. The electrode 13 comprises a distal tip 12 configured to deliver an electrical current to biological tissue and a proximal end 11 configured to be coupled to an energy source. The proximal end 11 of the electrode 13 may be sized and designed such that it may be operably coupled with an electrosurgical handpiece provided by a variety of different manufacturers. In some embodiments, the distal tip 12 is shaped in a manner conducive to cutting tissue such as in a blade shape. The length of the electrode 13 may vary depending on type of desired operation.

The electrode 13 is comprised of an electrically conductive material such as a metal or metal alloy. In certain embodiments, the electrode 13 is comprised of a stainless steel, such as 304 stainless steel. In some such embodiments, the electrode 13 consists of a stainless steel, such as 304 stainless steel. In order to facilitate improved self-cleaning capabilities, the distal tip 12 may have a non-stick coating applied, such as a PTFE layer or porcelain.

In some embodiments, the electrode 13 may be and/or include a monopolar blade electrode, a bipolar electrode, and/or the like. Additionally, or alternatively, the electrode 13 may be and/or include a plurality of electrodes.

With reference to FIGS. 1B and 1C, the electrode 13 is illustrated with a layer of insulation material 14 radially disposed on a middle portion of the electrode. The insulation material 14 is overmolded onto the electrode to form a body 15, which is shaped cylindrically to avoid snagging. The proximal end of the insulation material 14 is formed into a hexagonal shape in order to facilitate coupling with an electrosurgical handpiece. The distal tip 12 and proximal end 11 of the electrode are exposed (i.e., not covered by the insulation material).

The insulation material may be selected from materials having a relatively low dielectric constant, as compared to the device body material and the material comprising the electrode. In some embodiments, the insulation material comprises a polymer. In some such embodiments, the insulation material comprises PTFE.

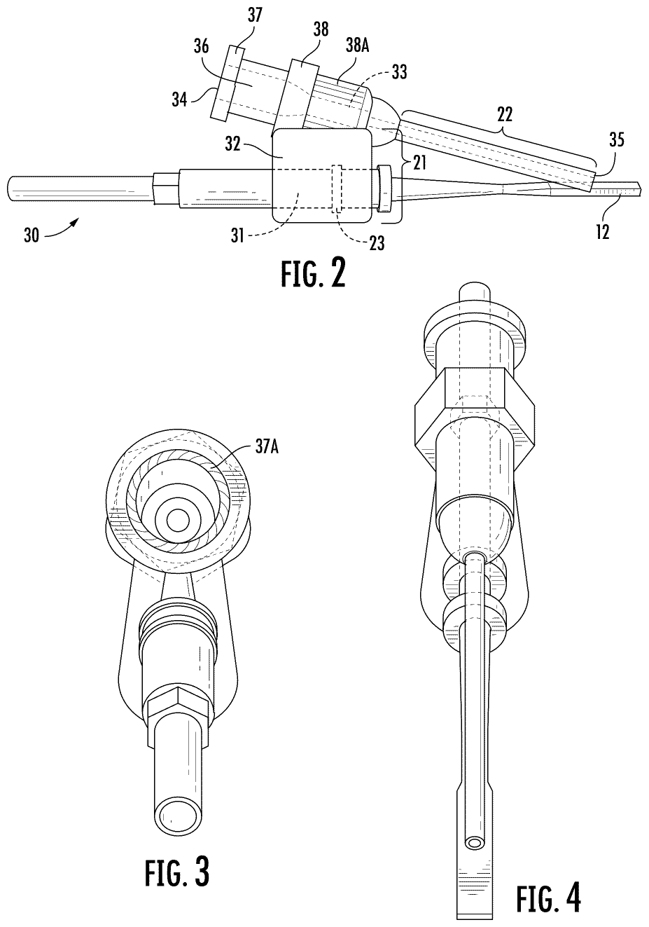

In various embodiments, additional texturing of the insulation 16A and 16B can be added to improve the connection between the insulated electrode (e.g., formed by the electrode 13 and the insulation material 14) and the electrocautery device body 32 (shown in FIG. 2) by improving the traction between the material forming the electrocautery device body and the insulating body. Texturing also assists the user in gripping the electrocautery device during tissue dissection.

With reference to FIGS. 2-4, an electrocautery device 30 is illustrated. The electrocautery device comprises an insulated electrode 31 and an electrocautery device body 32 having both a lower portion 21 that is radially disposed around the insulated electrode 31 and an upper portion 33 which is formed as a single unitary piece with the lower portion.

The upper portion 33 of the electrocautery device body comprises a first portion 34 at its proximal end and a second portion 22 at its distal end. The first portion 34 comprises a gas supply connection 37 that is couplable to a gas supply source. The gas supply source may be coupled to the gas supply connection directly or, alternatively, via a gas supply tube. In some embodiments, the gas supply connection includes threading 37A to accommodate gas supply sources that may be coupled via the threaded adapters of the gas supply connection. In some such embodiments (as depicted in FIG. 3), the threading is on an interior surface of the gas supply connection. Additionally or alternatively, the threading may be on an exterior surface of the gas supply connection. In some embodiments, the gas supply connection is tapered to create a friction-fit with a gas supply source, such as in a Luer Taper.

In some embodiments, the electrocautery device further comprises the gas tube and/or gas source. In such embodiments, the gas tube is permanently coupled to the gas supply connection of the electrocautery device via overmolding. For example, one or more materials may be overmolded over at least a portion of the overlapping sections of the gas tube and the gas supply connection. Alternatively, the gas tube may be permanently coupled to the electrocautery device by overmolding the gas tube with the device body material defining the gas supply connection. This connection may be formed at the time the electrocautery device body is initially formed. Alternatively, the gas tube may be permanently coupled to the electrocautery device by applying adhesive to the section where the gas tube is in contact with the electrocautery device body.

The second portion 22 is formed at the distal end of the upper portion and adjacent to the first portion. The second portion defines a gas delivery channel 36 having a gas tip 35 on its distal end. The gas tip is placed tangent to a flat side of the blade and rearward from the blade's distal tip to minimize the chance of blockage. The gas delivery channel 36 is in fluid communication with the gas supply connection 37 such that, when the electrocautery device is in use, gas flows through the gas delivery channel without obstruction and is delivered to the tissue adjacent the distal end of the electrocautery device. The second portion 22 is angled at its distal end. In some embodiments, the second portion is at an acute angle with respect to the distal tip 12 and disposed proximal to the distal tip 12 of the electrocautery device at a distance between 3 and 10 mm, thereby providing additional stability to the gas tip. In some embodiments, the second portion is disposed about 3 mm from the distal tip. In some embodiments, the second portion is disposed about 4 mm from the distal tip. In some embodiments, the second portion is disposed about 5 mm from the distal tip. In some embodiments, the second portion is disposed about 6 mm from the distal tip. In some embodiments, the second portion is disposed about 7 mm from the distal tip. In some embodiments, the second portion is disposed about 8 mm from the distal tip. In some embodiments, the second portion is disposed about 9 mm from the distal tip. In some embodiments, the second portion is disposed about 10 mm from the distal tip.

In some embodiments, the second portion is at an angle between 5 and 45 degrees with respect to the distal tip 12. In some embodiments, the outer diameter of the gas tip is less than or equal to the width of the distal tip in order to provide concentrated gas delivery to target structures.

In some embodiments, the gas tip may be formed by overmolding such as by compression molding or, alternatively, by injection molding. The gas tip may be formed from a translucent material to facilitate visualization and reveal potential blockages.

The gas tip 35 is positioned such that, as the gas is delivered, it is dispersed by the electrode's distal tip 12 in a linear pattern and in the direction of adjacent tissue that is not intended for dissection. In certain embodiments, the gas flow is constrained to one side of the gas tip. This adjacent tissue often includes fragile structures, such as vessels and nerves, which are gently pushed away from the electrocautery tip of the electrocautery device while the cauterizing tip maintains solid contact with the tissue. Additionally, when the electrocautery device is used for coagulation, the gas can help clear excessive blood from the surgical area while the blade edge opposite of the gas end maintains solid contact with the bleed site for coagulation as there is no direct gas flow that would inhibit contact. The gas keeps operative tissue relatively dry, increases the operative visibility, prevents char buildup on the blade and reduces the risk of fire in the operative field. Notably, in some embodiments, the gas delivery assembly is configured to deliver any fluid (i.e., a gas and/or a liquid).

The electrocautery device body may be formed by overmolding the device body materials over the insulated electrode 31. In some embodiments, the upper portion 33 and the lower portion 32 of the electrocautery device body are molded as a single unitary piece onto the insulated electrode 31. The electrocautery device body 32 is formed from a non-conductive material (e.g., plastic). In various embodiments, the electrocautery device body comprises one or more materials having properties that are more beneficial for gas delivery than the insulation material, such as a relatively high tensile strength. In certain embodiment, the device body material has a higher flexural modulus as compared to the first insulating material, because threading with a softer material such as an insulating material can allow the threaded gas tube to be over-rotated, resulting in damaged threading on the assembly and an unsecure gas connection. In certain embodiments, the device body material comprises ABS or polycarbonate. Additional valve body material 38 & 38A can be molded to improve the durability of the gas delivery assembly.

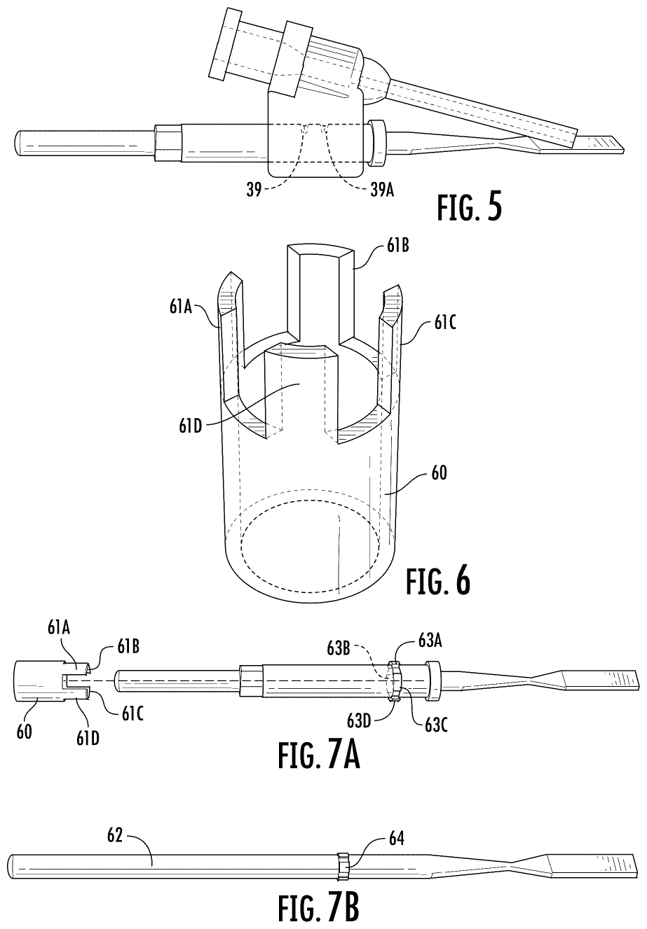

With reference to FIG. 5, another embodiment of the electrocautery device is illustrated. As shown in FIG. 5, the insulation material may be recessed to define notches 39 & 39A. This can be used in instances where the electrode insulating material and the material comprising the device body require a mechanical bond instead of a chemical bond.

In some embodiments, the electrocautery device body may be formed by injection molding the device body materials. In such embodiments, the device body has one or more cavities to accommodate the gas tip and/or electrode. Either or both of the gas tip and the electrode can be secured to the device body by applying adhesive to the one or more cavities after the gas tip and/or electrode are inserted into the one or more cavities.

In some embodiments, the electrocautery device may further comprise a buffer. A typical injection molding process requires that the insulated electrode be placed within a mold cavity with molten plastic. During this process, it is possible that the insulation material will become deformed in response to the high temperature of the molten plastic, resulting in decreased structural integrity. In order to maintain the structure of the insulating material, a buffer may be placed around the insulated electrode to inhibit deformation of the insulation material. The buffer material may include but is not limited to thermoplastics, metals, and wood. The buffer physically constrains the insulation to its current shape and reduces heat transfer to the insulation material on the electrode, helping the electrode insulation retain its structure. The buffer may fully or partially cover the insulation material. In some embodiments, the buffer may have an inner diameter slightly greater than the diameter of the insulating material of the electrode such that the buffer is friction fit with the electrode insulation. In alternative embodiments, the insulated electrode may comprise notches, texturing, and/or other characteristics for inhibiting movement of the buffer.

With reference to the embodiment depicted in FIGS. 6, 7A and 7B, a buffer 60 is depicted. The buffer may have one or more lugs 61A, 61B, 61C, 61D on one or both ends that are configured to couple the buffer to the insulation material of the insulated electrode 62. The insulation material of the insulated electrode may define anti-rotation tabs 63A, 63B, 63C, 63D which, when coupled to the lugs 61A, 61B, 61C, 61D, inhibit movement of the buffer. The electrode 62 may optionally be shaped to partially define the anti-rotation tabs. For example, the electrode may be shaped to include fins 64, as shown in the electrode of FIG. 7B. When the fins 64 are overmolded with an insulation material, the insulation material will conform to the fins of the electrode, to form the anti-rotation tabs. In alternative embodiments, the electrode may comprise notches, texturing, and/or other characteristics for inhibiting movement of the buffer.

With reference to FIG. 8, an electrocautery device having a buffer disposed around the insulation material is depicted. The device body material is overmolded onto the insulated electrode and the buffer, forming a mechanical bond between the buffer, insulation, and device body materials.

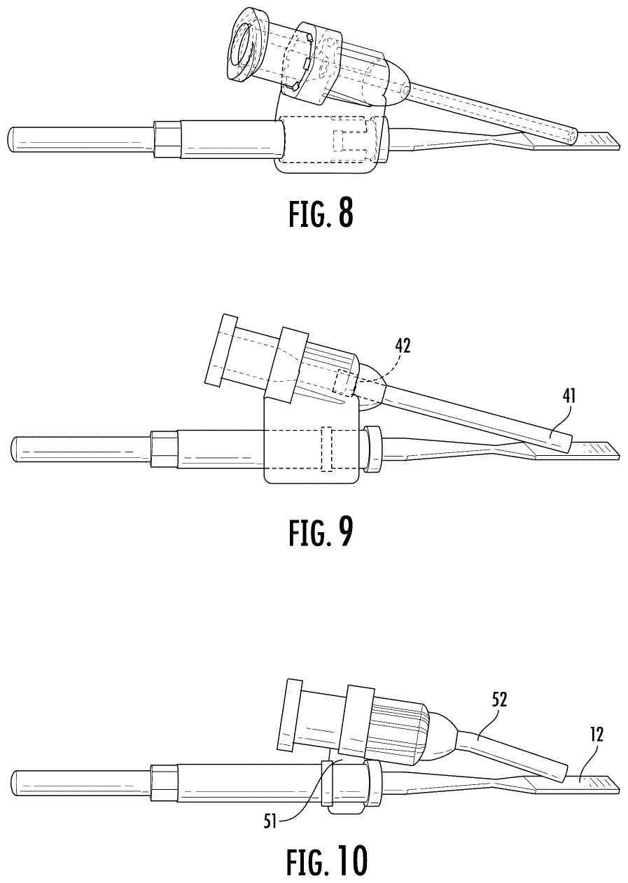

With reference to FIG. 9, an electrocautery device in which the gas tip 41 is formed from a third material that is distinct from the device body material and the insulation material is depicted. The gas tip 41 is formed by overmolding and is shaped with a neck 42 to enhance the bond between the device body material and the gas tip. In some embodiments, the gas tip has notches on its upper potion. In such embodiments, the notches are held in place by the neck 42, thereby preventing de-coupling of the gas tip from the device body.

Use of a distinct third material to form the gas tip may be advantageous when the properties of the third material (e.g., strength, heat resistance, and/or the like) are preferred over the properties of the device body material. The gas tip may be comprised of a metal, such as stainless steel. In some embodiments, the gas tip may be welded to the distal tip. Optionally, the third material forming the gas tip may be coated with a non-stick coating, such as a PTFE or porcelain coating. In some embodiments, the gas tip 41 is formed by compression molding. In an alternative embodiment, the gas tip 41 is formed by injection molding.

With reference to FIG. 10, an electrocautery device having a lower profile body 51 is depicted. The distal second portion 52 of the upper portion of the valve body is canted at an angle such that the overall length and height of the gas delivery assembly can be reduced while maintaining the acute angle and distance rearward in relation to the distal tip 12 of the electrode. In various embodiments, the gas delivery channel may extend parallel to the distal tip, with the distal tip 12 of the electrode. In some embodiments, the gas delivery assembly may be canted at an angle to achieve the desired acute angle in relation to the electrocautery blade.

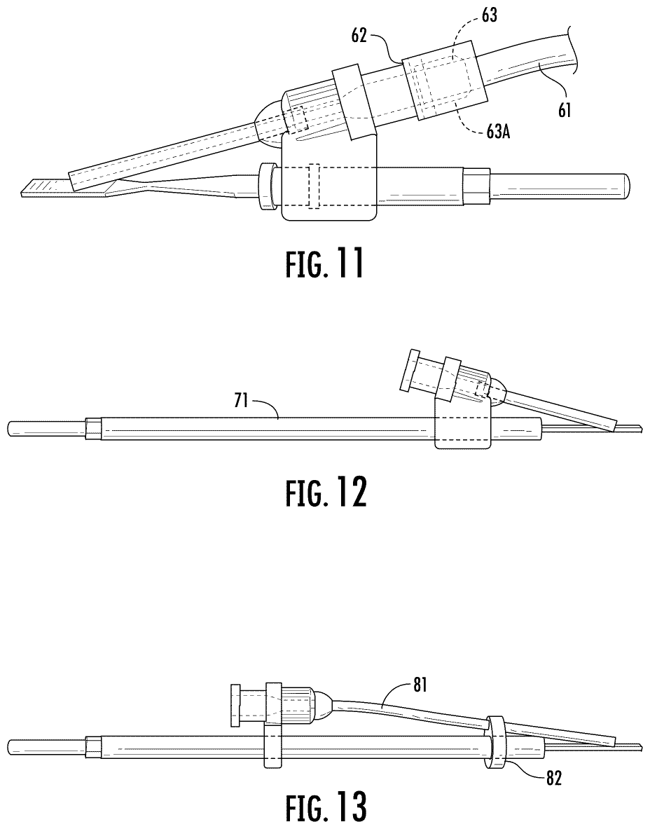

With reference to FIG. 11, another embodiment of the electrocautery device is depicted in which a gas tube 61 is fluidly coupled to the assembly. The gas tube 61 is overmolded to the assembly, and a catch 62 to hold the gas tube 61 in place and prevent movement when gas is provided from the gas supply source. Fins 63 & 63A are overmolded onto the gas tube to help stabilize the tube within the gas supply connection of the electrocautery device. In some embodiments, the gas tube 61 may be permanently coupled to the electrocautery device. In such embodiments, the gas tube is permanently coupled to the gas supply connection of the electrocautery device via overmolding. In some such embodiments, the gas tube is permanently coupled to the electrocautery device by overmolding one or more materials over at least a portion of the connected portion of the gas tube and gas supply connection. In other embodiments, the gas tube is permanently coupled to the electrocautery device by overmolding the gas tube with the device body material defining the gas supply connection. In other embodiments, the gas tube may be removable. In other embodiments, the gas tube is permanently attached with the use of adhesive. In some embodiments, the gas tube is held in place via friction with a cavity at the gas supply connection.

With reference to FIG. 12, an electrocautery device having an extended length is depicted. In this embodiment, the electrode 71 is elongated and the gas tip of the gas delivery assembly is at an acute angle at a distance that is disposed 3 mm to 10 mm from the distal tip of the electrode of the electrocautery device, in the proximal direction.

With reference to FIG. 13, another embodiment of the electrocautery device having an extended length is depicted. In this embodiment, the upper portion comprises an elongated tube 81 defining the gas delivery channel. The elongated tube 81 is canted to reduce the body size and vertical profile of the electrocautery device. An additional section 82 is overmolded to the elongated tube and the insulation of the electrocautery blade to increase the stability of the elongated tube.

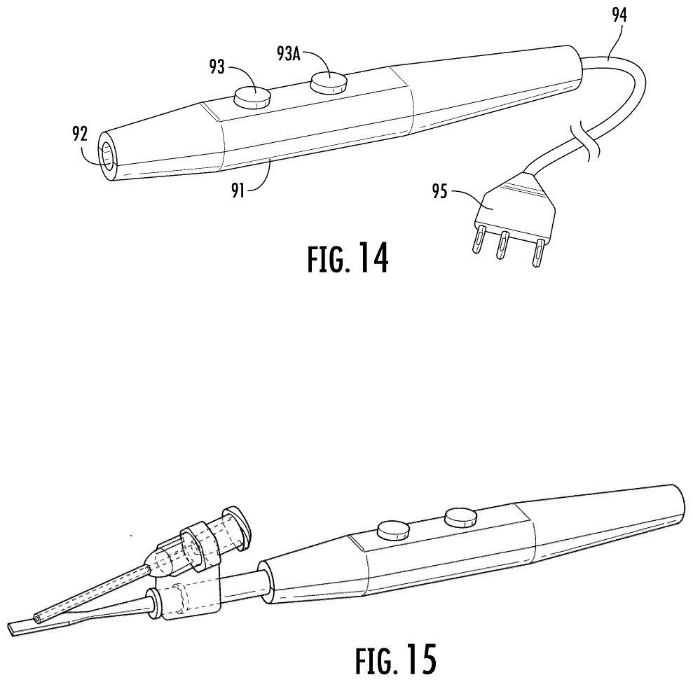

With reference to FIG. 14, an isometric view of an electrosurgical handpiece is depicted. In this embodiment, the electrosurgical handpiece comprises a housing 91 that is normally gripped by the operator, a slot 92 which internally contains an electrode fastener which holds the electrode, buttons 93 and 93A which control the delivery of current to the electrode, and electric wire 94 which is connected to a plug 95 which is intended to connect to an energy supply source such as an electrosurgical generator.

The handpiece allows a user to control the amount of power supplied to the electrode and, ultimately, the biological tissue. The optimal amount of power to be supplied is dependent upon factors such as the size and width of the target tissue, in addition to the desired procedure. The “cutting” mode is typically used for dissecting muscle, subcutaneous fat and fascia or membrane tissue. When the electrocautery device is used in cutting mode, between 40 and 100 watts are applied to the tissue. The “coagulation” mode is typically used to stop bleeding around muscle or subcutaneous fat, but it may also be used for cutting tissue near vessels and nerve bundles and more sensitive fascia or membrane tissue. When the electrocautery device is used in coagulation mode, between 20 and 60 watts are applied to the tissue.

With reference to FIG. 15, the electrocautery device coupled with an electrosurgical handpiece is depicted. In this embodiment, the lower portion of the electrocautery device body is coupled to a slot in the electrosurgical handpiece and held in place by an internal electrode fastener within the handpiece. The overmolded electrode with gas delivery assembly may be rotated in relation to the handpiece.

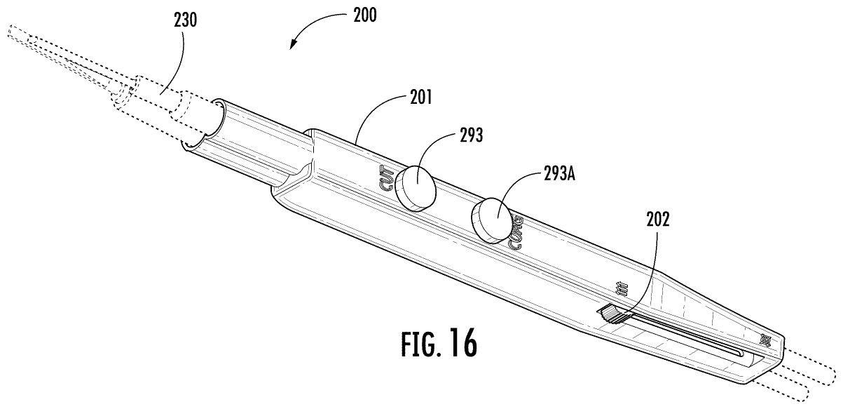

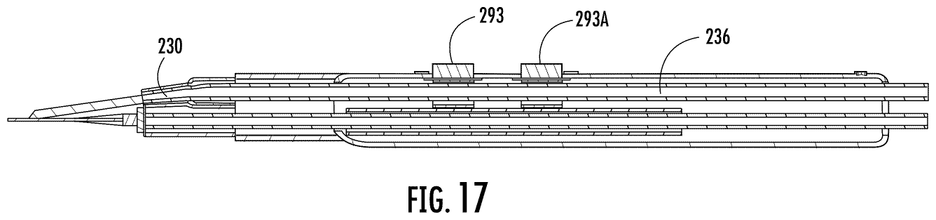

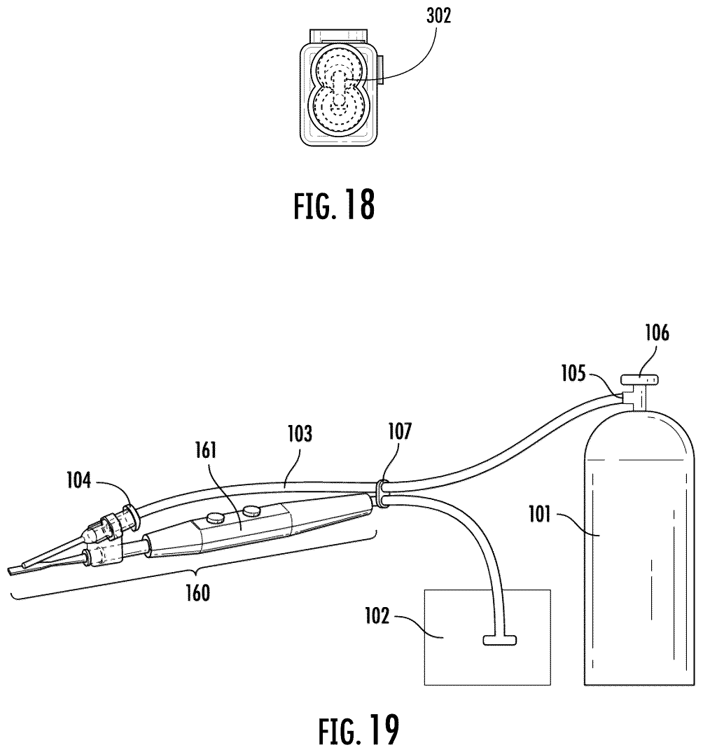

With reference to FIGS. 16-18, various views of an example electrocautery handpiece 200 in accordance with one or more embodiments of the present disclosure, is illustrated. As shown, the electrocautery handpiece 200 may include a housing 201 that defines an internal cavity that at least partially receives an electrocautery device therein. By way of example the housing 201 may be dimensioned (e.g., sized and shaped) so as to at least partially receive the electrocautery device 230 therein. Although described hereinafter with reference to the electrocautery device 230 of FIGS. 1-15, the present disclosure contemplates that the electrocautery handpiece 200 may be configured for use with other electrocautery devices, surgical tools, and/or the like based on the intended application of the handpiece 200. With continued reference to FIGS. 16-18, the electrocautery handpiece 200 may further include one or more electricity flow controller(s) 293, 293A and one or more gas flow controllers 202.

The gas flow controller 202 may, in some embodiments define a roller 202 that may be configured such that a user may control the flowrate at which a gas is delivered by the electrocautery device by manipulating the roller. In some embodiments, actuation of the roller in a first direction causes the roller to exert pressure on the gas delivery channel 236, thereby selectively restricting the flow of gas by decreasing the cross-sectional area of the lumen within the gas delivery channel. The restriction of gas flow is reversible by actuation of the roller in a second direction that is opposite the first direction. The degree by which the gas flow is through the gas delivery channel is restricted may be incrementally adjusted, based on the distance the gas flow controller is actuated in the first direction. In some embodiments, the handpiece is designed to restrict the roller's movement in the first direction such that it is unable to completely seal the gas delivery channel. This restriction ensures that a small amount of gas is permitted to flow through the gas delivery channel, thereby preventing a pressure build-up in the gas delivery channel. In some embodiments, the external portion of the gas flow controller has a first position and a second position. In such embodiments, the gas flow controller is configured to prohibit the flow of gas when in the first position and allow the flow of gas when the external portion of the gas flow controller is in the second position. In some embodiments, roller clamps, pinch clamps, stopcocks, or other fluid control mechanisms known in the art may be used to restrict air flow through the gas delivery channel. In still other embodiments, gas flow may be controlled by manipulation of an occlusive device which occludes the lumen of the gas delivery channel to restrict the volume of gas flow through the tube. In some embodiments, the gas flow control mechanism may be actuated or manipulated via a user interface integrated into the handpiece.

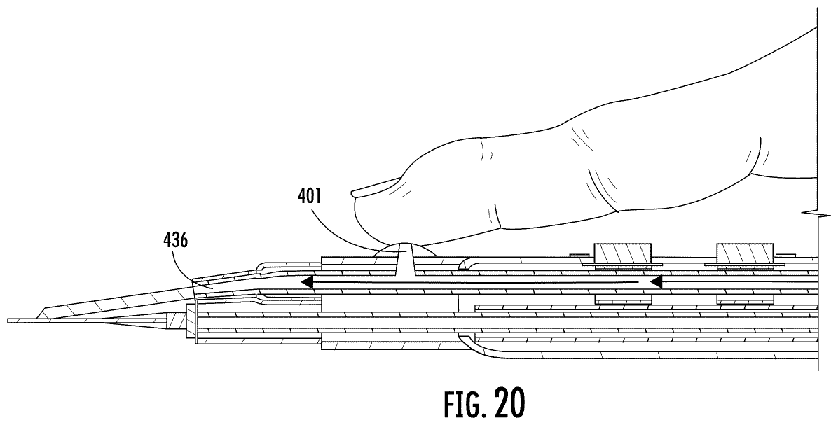

With reference to FIG. 20, in some embodiments, the electrocautery device comprises a gas aperture 401 in fluid communication with the gas delivery channel 436. When the gas aperture is unobstructed, gas is permitted to flow out of the gas delivery channel through the gas aperture. When the gas aperture is obstructed (e.g., by a user positioning a hand or finger over the gas aperture, as depicted in FIG. 20), gas flow through the gas aperture is restricted and therefore the flow of gas is directed through the gas delivery channel. In some embodiments, the gas aperture may extend perpendicularly from the gas delivery channel.

The electricity flow controllers 293 and 293A, in some embodiments define buttons 293 and 293A, having external portions that extend through an electricity flow controller opening in the housing and internal portions that are disposed within the internal cavity of the housing such that, when a user actuates the electricity flow controllers (e.g., by depressing the external portion thereof), the buttons contact an electricity control unit 301, thereby permitting the flow of electricity. In some embodiments, each of the electricity flow controllers is formed to define an opening 302 through which the gas delivery channel 236 of the electrocautery device may pass.

In an alternative embodiment, the electricity flow controller may be a variable control mechanism. For example, a knob or sliding mechanism may be used to allow a user to gradually increase the flow of electricity to the electrode. In some embodiments, the electricity flow controllers may be integral with a user interface configured to allow a user to increase the electricity provided to the electrode by interacting with the user interface.

The electricity control unit 301 may be configured to supply different amounts of electricity to the electrocautery device based on which of the electricity flow controllers 293, 293A is actuated by the user. For example, manipulation of one button would supply an optimal amount of electricity for electrical cutting, while manipulation of another button would supply an optimal amount of energy for coagulation. The electricity control unit 301 may be isolated using an insulating material. In some embodiments, the electricity control unit comprises a circuit socket; a printed circuit board (PCB), including an AC and/or DC circuit; and wiring, including at least a portion of the electric wire 294. However, one having ordinary skill in the art will recognize that the electricity control unit may comprise a variety of electrical components and configurations.

In some embodiments, the gas delivery channel 236 may extend through the entire length of the electrocautery handpiece. However, in alternative embodiments, the gas delivery channel may extend through only a portion of the electrocautery handpiece. In such embodiments, the electrocautery device may comprise additional components for gas delivery (e.g., a plastic barb) which may be coupled to or disposed within the electrocautery handpiece. The gas delivery channel may be coupled to a pressure relief valve to prevent excess gas from accumulating in the gas delivery channel. In some embodiments, the gas source comprises a regulator (or, alternatively, may be coupled to a regulator) designed to ensure that the gas is provided at the user's preferred pressure level. In some embodiments, the regulator further includes a pressure relief valve to prevent excess pressure from gas accumulation within the gas delivery channel.

In some embodiments the electrocautery device is removably coupled to (i.e., detachable from) the housing 201 of the handpiece (e.g., via friction fit). In such embodiments, the electrocautery handpiece may comprise fittings for coupling the electrocautery device to the electrocautery handpiece. In an alternative embodiment, one or more components of the electrocautery device may be permanently fixed to the housing of the electrocautery handpiece. For example, the electrode of an electrocautery device may be fixed to the housing using an adhesive and/or by overmolding.

In some embodiments, the housing 201 of the electrocautery handpiece may be formed from a transparent material (e.g., a transparent polymer) to allow the user to visually evaluate the internal components and identify potential blockage, damage and/or contamination within the electrocautery handpiece or the electrocautery device. In some embodiments, the handpiece is formed from a rubber polymer to increase the ease with which the user is able to grip the handpiece.

With reference to FIG. 19, the system for dissection of biological tissue is depicted. The system comprises an electrocautery device 160, a gas supply source 101 coupled to a gas supply of the electrocautery device via a gas supply tube 103, an energy supply source 102 configured to deliver an electrical current to the electrode of the electrocautery device, and a handpiece 161 that is operably coupled to the energy supply source.

At a first end, the gas supply tube 103 connects to the electrocautery device 104 via the gas supply connection of the electrocautery device. At a second end, the gas supply tube 103 connects to a nozzle 105 on the gas source. In various embodiments, the gas supply tube is fluidly connected to one or both of the electrocautery device 104 and gas source nozzle 105 via threaded adapters. A valve on the gas source 106 may be used to control the flow of gas to the electrocautery blade.

The gas supply source may be a gas tank or other feed such as a box or regulator. Alternatively, the gas supply source may be a direct gas line. Alternative gas control systems may be used. Examples of such alternative gas control systems include a foot pedal valve control, a remote valve located next to the user of the electrocautery device, as well as any other gas control system. In some embodiments, the gas supplied by the gas supply source is an inert gas. In some such embodiments, the gas is carbon dioxide. In other embodiments, the gas supplied by the gas supply source is argon.

The gas tube and gas supply source are not coupled to the energy supply source. Decoupling of the gas supply source and the energy supply source isolates damage caused by failure of either system. A coupler 107 is attached to the gas tube and the electric wire to keep the system organized and avoid potential injury to the use or damage to the system.

In some embodiments, the entirety of the gas supply source and gas tube is external to the handpiece and visible to the operator. This allows for the identification of potential blockages or damage to the gas system.

There have been illustrated and described various embodiments of an overmolded electrocautery blade with gas delivery assembly. The features of the several embodiments described herein may be combined without limitation.

Modifications of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An electrocautery handpiece comprising:
an electrocautery device;
a housing defining an internal cavity, wherein the internal cavity is configured to at least partially receive the electrocautery device therein;
one or more gas flow controllers having an external portion that extends through a gas flow controller opening in the housing and an internal portion that is disposed within the cavity of the housing and configured to selectively restrict a gas flow through a gas delivery channel of the electrocautery device;
one or more electricity flow controllers having an external portion that extends through an electricity flow controller opening in the housing and an internal portion that is disposed within the internal cavity of the housing; and
an electricity control unit operably coupled to the one or more electricity flow controllers, wherein, in an instance in which the external portion of the one or more electricity flow controllers electric flow controllers is actuated by a user, the electrical control unit is configured to supply electricity to the electrocautery device.

2. The electrocautery handpiece of claim 1, wherein at least one of the one or more gas flow controllers is configured to, when actuated by the user in a first direction, exert pressure on the gas delivery channel of the electrocautery device such that the gas flow through the gas delivery channel is decreased.

3. The electrocautery handpiece of claim 2, wherein the gas flow controller is configured to:
prohibit the flow of gas through the gas delivery channel when the external portion of the gas flow controller is in a first position, and
allow the flow of gas through the gas delivery channel when the external portion of the gas flow controller is in a second position.

4. The electrocautery handpiece of claim 2, wherein the gas flow through the gas delivery channel is varied by modifying a distance of the external portion of the gas flow controller in the first direction.

5. The electrocautery handpiece of claim 4, wherein the external portion of the gas flow controller is a roller.

6. The electrocautery handpiece of claim 4, wherein the gas flow controller opening is dimensioned to limit actuation of the gas flow controller in the first direction such that the gas flow controller is prevented from precluding gas flow through the gas delivery channel.

7. The electrocautery handpiece of claim 1, wherein the one or more of the gas flow controllers further comprise an occlusive element configured to occlude the gas delivery channel such that the gas flow through the gas delivery channel is decreased.

8. The electrocautery handpiece of claim 1, wherein the electrocautery handpiece further comprises a pressure relief valve operably coupled to the gas delivery channel of the electrocautery device.

9. The electrocautery device of claim 1, wherein:
the gas delivery channel is a gas tube; and
the internal portion of the one or more electricity flow controllers defines an opening dimensioned to allow the gas tube to extend therethrough.

10. The electrocautery handpiece of claim 1, wherein the housing is configured to be removably coupled to one or more components of the electrocautery device via fittings disposed within the internal cavity of the housing.

11. The electrocautery handpiece of claim 1, wherein the housing is permanently fixed to at least a portion of the electrocautery device.

12. The electrocautery handpiece of claim 1, wherein at least one of the one or more electricity flow controller comprises buttons configured to, when actuated by the user, manipulate the electrical control unit such that the electrical control unit supplies electricity to the electrocautery device.

13. The electrocautery handpiece of claim 1, wherein the external portion of at least one of the one or more electricity flow controllers comprises an electricity control mechanism configured to allow the user to adjust an amount of electricity supplied to the electrocautery device.

14. A surgical device for controlled dissection of biological tissue, the device comprising:
an electrocautery device comprising:
an insulated electrode having a distal tip configured to deliver an electrical current to a biological tissue, and
a gas delivery assembly comprising:
a gas delivery channel configured to be operably coupled to a gas supply source via a gas supply connection, and
a gas tip coupled to and extending distally from the gas delivery channel; and
an electrocautery handpiece operably coupled to the electrocautery device, the electrocautery handpiece comprising:
a housing,
an electricity control unit configured to control the flow of electricity from an electricity source to the electrocautery device,
one or more electricity flow controllers operably coupled to the electricity control unit such that the flow of electricity to the electrocautery device is varied via the one or more electricity flow controllers,
one or more gas flow controllers operably coupled to the gas delivery assembly of the electrocautery device such that the flow of a gas in the gas delivery channel of the electrocautery device is varied via the one or more gas flow controllers.

15. The surgical device of claim 14, wherein the gas flow controller is configured to, when actuated by the user in a first direction, selectively restrict a gas flow through the gas delivery channel of the electrocautery device.

16. The surgical device of claim 15, wherein the gas flow controller is configured to, when actuated by a user in a first direction, exert pressure on the gas delivery channel of the electrocautery device such that the gas flow through the gas delivery channel is decreased.

17. The surgical device of claim 16, wherein the gas flow through the gas delivery channel is varied by modifying the distance of the external portion of the gas flow controller in the first direction.

18. The surgical device of claim 15, wherein the one or more gas flow controllers further comprise an occlusive element configured to occlude the gas delivery channel such that the gas flow through the gas delivery channel is decreased.

19. The surgical device of claim 14, wherein at least one of the one or more electricity flow controller comprises buttons configured to, when actuated by the user, manipulate the electrical control unit such that the electrical control unit supplies electricity to the electrocautery device.

20. The surgical device of claim 14, wherein the electrocautery device is removably coupled to the electrocautery handpiece.
